(19) United States
(12) Patent Application Publication
(10) Pub. No.: US 2023/0071741 A1
(43) Pub. Date: Mar. 9, 2023

(54) TRANSIENT DISRUPTION OF THE BLOOD-RETINAL BARRIER OF A HUMAN AND USES THEREOF FOR TREATING A RETINA DISORDER

(71) Applicants: Sorbonne Universite, Paris (FR); Assistance Publique - Hôpitaux de Paris, Paris (FR)

(72) Inventor: Alexandre Carpentier, Paris (FR)

(21) Appl. No.: 17/984,802

(22) Filed: Nov. 10, 2022

Related U.S. Application Data
(62) Division of application No. 16/609,385, filed on Oct. 29, 2019.

(30) Foreign Application Priority Data
May 4, 2017 (EP) 17305502.1

(51) Int. Cl. A61F 9/00; A61M 37/00

(52) U.S. Cl. CPC A61F 9/0017; A61M 37/0092; A61M 2210/0612

(57) ABSTRACT

The present invention relates to an ultrasound contrast agent for use in treating a retina disorder by transiently disrupting the blood-retinal barrier (BRB) of a human, wherein the ultrasound contrast agent is administered just before and/or during the application, to the retina of the human, of an unfocused ultrasound (US) beam. The present invention further relates to a therapeutically active agent for use in treating a retina disorder in a human, wherein the therapeutically active agent is to be delivered in combination with an ultrasound contrast agent, which is administered before and/or during the application, to the retina of the human, of an unfocused ultrasound (US) beam in order to transiently disrupting the blood-retinal barrier (BRB) of the human, to allow the therapeutically active agent to cross the BRB and to target the retina. The present invention also relates to an eye ultrasound delivery device that may be used to treat a retina disorder.

### CROSS-REFERENCE TO RELATED APPLICATIONS

The present application is a divisional of copending U.S. patent application Ser. No. 16/609,385, filed on Oct. 29, 2019, which is the National Stage of International Application No. PCT/EP2018/061592, filed May 4, 2018, which claims priority to European Application No. 17305502.1, filed May 4, 2017. The contents of the prior applications are hereby incorporated by reference herein in their entirety.

### TECHNICAL FIELD

The present invention relates to means for transiently disrupting the blood-retinal barrier of a human. More particularly, the invention relates to ultrasound contrast agent for use in treating a retina disorder, wherein the ultrasound contrast agent is used in combination with unfocused ultrasound beam for transiently disrupting the blood-retinal barrier. The invention further relates to an eye ultrasound delivery device suitable for disrupting the blood-retinal barrier.

### BACKGROUND

The eye is a well-protected organ that possesses several barriers for forbidding foreign substances to enter into the ocular tissues. These natural barriers protect the eye from external aggressions. However, they also limit the access of drugs to target tissues of the eye, and thereby the possibilities to treat retinal disorders, such as degenerative retinopathies, diabetic retinopathies, etc. Up to now, the common routes for delivering ocular drugs include intravitreal, topical and systemic approaches.

Intravitreal injections, where a drug is directly injected into the vitreous of the eye, is the most effective route, and allow to deliver drugs to the fundus. However, this method is unpleasant, invasive and increases the risk of retinal detachment, retinal hemorrhage, and/or glaucoma for the patient. Furthermore, for chronic diseases requiring repeated intraocular injections, the risks multiply. Topical delivery consists in applying a liquid or a gel on the surface of the eye, with the goal of drug penetration through the cornea or sclera. However, the penetration of the drug through this route is low. Indeed, the drug may be eliminated by solution drainage, lacrimation and tear dilution, conjunctival adsorption, or blocked by the tight junctions in the corneal epithelium. Even after reaching the first tissues, the drug may still be removed by intraocular tissues and fluids. As a result, only 3% of the administered dose eventually target the retina. Since the penetration is low, efforts are still being made on the pharmaceutical agent properties to optimize their penetration. The systemic intravenous drug delivery technique is the worse efficient and the worse selective drug delivery technique, due to the drug dilution in the whole body and the really poor penetration in the retina due to the presence of the blood-retinal barrier (BRB) that limit the access of the drugs to the retina. A large systemic dose of drug is necessary to reach the therapeutic level because the BRB dramatically decreases the flux of drugs to the retina. However, undesirable side effects are resulted when a high concentration of therapeutics is distributed in the body via the circulation system. The BRB, which is formed by complex tight junctions of the endothelium of retinal blood vessel and retinal pigment epithelium, is a functional impediment for drug delivery from the peripheral circulation to the retina. Currently, the most part of clinically validated drugs with potential therapeutic effects for retinal disorders cannot cross the BRB.

Recently, a less invasive method has been developed using ultrasounds, based on the fact that ultrasounds were already explored in enhancing drug delivery in transdermal route. Accordingly, ultrasounds were used to increase drug diffusion in the vitreous humor. As an example, the permeability of the cornea may be increased using the principle of ultrasound mechanical oscillation of the cornea. The drug penetration of a drug topically applied on the cornea, may be increased of 2.8 times (Nabili M et al J Ther Ultrasound (2014); Zderic V et al, Cornea (2004)). However, this technique induces consistent mechanical shear stress and damageable side effects on the retina, including cell disunion, and severe visual deficit (Lafond M et al, Expert Opin Drug Deliv. (2016). A technique called microstreaming has also been developed. This technique is based on ultrasound mechanical oscillation of the vitreous humor liquid, which leads to spontaneous formation of microbubbles (cavitation). Micrometric flows are created around the locally ultrasound-created oscillating bubbles, improving the diffusion and penetration of drugs locally present.

However, even if ultrasounds allow a better drug diffusion within the eye, this is largely insufficient since the diffusion is solely doubled from the 3% baseline uptake to 6%. In addition, associated hyperthermia may lead to coagulation and thereby destroy specific region of the eye.

Therefore, there is still a need for a non-invasive, safe and effective system for increasing the drug delivery to the eyes of a human.

### SUMMARY

The present invention provides a novel, non-invasive, safe and effective ultrasound way to enhance the delivery of a drug to the eye from the systemic circulation. More particularly, the present invention proposes a method to transiently disrupting the BRB, in order to allow a drug present in the systemic circulation to cross the BRB and to target the retina. More particularly, according to the invention, the BRB is disrupted by combining intravenous administration of an ultrasound contrast agent and application of an unfocused ultrasound (US) beam to the retina. The use of unfocused US beams allows to cover a large part of the retina's surface in a short period of time and thereby to disrupt a large part of the BRB. The disruption of the BRB allows passage of otherwise non-permeable drugs to the retinal tissue.

The present invention therefore relates to an ultrasound contrast agent for use in treating a retina disorder by transiently disrupting the blood-retinal barrier of a human, wherein the ultrasound contrast agent is intravenously administered, before and/or during the application, to the retina of the human, of an unfocused ultrasound beam.

The BRB disruption allows to molecules already present in the systemic circulation, such as growth factors, antibodies, or the like to reach the retina and thereby to have a prophylactic or therapeutic effect thereon.

The present invention further relates to a therapeutically active agent for use in treating a retina disorder in a human, wherein the therapeutically active agent is to be delivered in combination with an ultrasound contrast agent, which is administered before and/or during the application, to the retina of the human, of an unfocused ultrasound beam in order to transiently disrupting the blood-retinal barrier of the human, to allow the therapeutically active agent to cross the BRB and to target the retina.

The BRB disruption allows to molecules that were injected in the systemic circulation, to reach the retina and thereby to have a prophylactic or therapeutic effect thereon.

Advantageously, at least two US beams are applied sequentially to the retina, to favor the opening of the BRB.

The present invention further relates to an Eye ultrasound delivery device, suitable to be used with ultrasound contrast agent according to anyone of the previous claims, comprising at least two unfocused US transducers disposed on a substrate suitable to be applied on the cornea of a human, wherein the unfocused US transducers can be activated sequentially. To this end, any means allowing sequential activation of the unfocused US transducers may be used. For instance, the unfocused US transducers may be controlled by separate activation means, or they may be controlled by same activation means that are programmed to activate said unfocused US transducer sequentially. The sequential activation of the different transducers, allows to avoid ultrasound beams superposition. The unfocused US transducers are preferably unfocused planned transducers.

Further features of the present invention will be apparent from the accompanying drawings and the following detailed description.

### BRIEF DESCRIPTION OF THE DRAWINGS

FIGS. 1A and 1B illustrate a method for transiently disrupting the BRB of a human using an eye ultrasound delivery device of the invention. The eye ultrasound delivery device comprises one central unfocused planned US transducer, and four peripheral unfocused US transducers, equidistant from each other's, placed sufficiently lateral to avoid ultrasound drawback on iris. A security non emission zone is utilized for monitoring passive transducer.

FIG. 2 illustrates another method for transiently disrupting the BRB of a human using an eye ultrasound delivery device comprising concentric four unfocused plan US transducers emitting sound waves at different frequencies. A: Ultrasound waves from US transducers 20 and 21; B: Ultrasound waves from US transducers 22 and 23; C: Sum of the ultrasound waves A and B; “s”: Pressure threshold above which disruption of the BRB is observed.

### DETAILED DESCRIPTION

Despite the pharmaceutical industry develops efficient drugs to treat retinal disorders, most of them are unable to reach the retina due to the impermeability of BRB. Furthermore, it is known that several retinal disorders would be avoided or reduced if the molecules developed by the immune system could easily target the tissues of the eye. The impermeability of the BRB has been identified as the main barrier for many treatments of the retina in human. In this context, safe and transiently disruption of the BRB has been proven to present a significant interest in eye's treatment.

The invention therefore relates to ultrasound contrast agent for use for transiently disrupting the BRB of a human, wherein the delivery of ultrasound contrast agent is combined with the application of ultrasound beam(s) to the retina of the human. Uses thereof for delivering substances into the retina of the subject and/or for treating a retina disorder are also described herein. It is also an object of the present invention to provide a method for safely opening the BRB of a human using application of US beams on the retina, combined with intravenous administration of an ultrasound contrast agent.

The present disclosure will best understood by reference to the following definitions.

### Definitions

In the context of the invention, the term “retinal” refers both to the anatomical structure of the eye containing photosensitive neurons and ganglial cells, and its supportive underneath structure called choroid tissue.

The term “Blood retina barrier” or “BRB” refers both to the blood barrier of the strict retinal layer vessels and the blood barrier of the choroid vessels.

In the context of the invention, the term “disrupting the BRB”, “opening the BRB” or “increasing the permeability of the BRB” are used to refer to an increased susceptibility of the BRB to the passage of molecules there through that occurs without detectable damages of the retinal and/or choroid tissue.

The term “ultrasound contrast agent” is used herein to refer to a substance (solid, liquid or gas) that is able to enhance the contrast between the region containing the agent and the surrounding tissue in an ultrasound image. Advantageously, the ultrasound contrast agent corresponds to small bubbles of a gas, termed “microbubbles,” with an average diameter between 1 μm and 20 μm. Said microbubbles oscillate and vibrate when US is applied and may reflect ultrasound waves. The ultrasound contrast agent is generally injected intravenously into the blood stream, wherein it remains for a limited period of time.

The term “ultrasound beam”, “ultrasound wave” and “ultrasound” are used indifferently for designating sound waves with frequencies higher than 200 kHz. The ultrasound emission is unfocused due to the use of plan transducer(s). Ultrasound are described as “unfocused ultrasound”.

As used herein, “subject” refers to a “human”, i.e., a person of the species Homo sapiens, including man, woman, child and human at the prenatal stage. In one embodiment, a subject may be a “patient” who is awaiting the receipt of, or is receiving medical care or was/is/will be the object of a medical procedure, or is monitored for the diagnosis or the development of a disease.

In the context of the invention, the terms “treatment”, “treat” or “treating” are used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies.

A “therapeutically effective amount” or “efficient concentration” refers to mean levels or amount of substance that is aimed at, without causing significant negative or adverse side effects to the target, delaying or preventing the onset of a disease, disorder, or condition related to a retina disorder; slowing down or stopping the progression, aggravation, or deterioration of one or more symptoms of a retina disorder; bringing about ameliorations of the symptoms of the disease, disorder, or condition related to a retina disorder; reducing the severity or incidence of a retina disorder; or curing a retina disorder. A therapeutically effective amount may be administered prior to the onset of the retina disorder, for a prophylactic or preventive action. Alternatively or additionally, the therapeutically effective amount may be administered after onset of the disease, for a therapeutic action.

Throughout the disclosure, various aspects of the invention can be presented in a range format. It should be understood that the description in range format is for convenience and brevity and should not be constructed as an inflexible limitation on the scope of the invention. Accordingly, the description of a range should be considered to have specifically disclosed all the possible subranges as well as individual numerical values within that range, range values being included.

### Application of Ultrasound Beams

The BRB may be disrupted by both applying an unfocused ultrasound (US) beam to the retina of a human subject, and administering intravenously an ultrasound contrast agent.

According to the invention, the unfocused US beams are applied to the retina of the subject, with a pressure level ranging from 0.3 to 2 MPa. In the context of the invention, the “pressure level” refers to the maximum acoustic pressure measured in the acoustic field of the emitter in water. Advantageously, the unfocused US beams are applied within a pressure range of 0.7 MPa to 1.25 MPa, preferably within a pressure range of 0.8 MPa to 1.1 MPa.

In the context of the invention, the value of the pressure level corresponds to the value of the pressure level onto the retina. In particular embodiment, the pressure coming out of the emitter may be higher, in order to take into account attenuation due to intervening tissues. Generally speaking, such attenuation may be at most of 30%.

According to the invention, the resonance frequency of the unfocused US beam preferably ranges from 0.5 to 3 MHz. In a particular embodiment, the frequency of the unfocused US beam is approximately 1 MHz. In another particular embodiment, the frequency of the unfocused US beam is approximately 2 MHz.

In an embodiment, the unfocused US beam is applied in pulses of duration ranging from 10 to 300 ms and with a pulse repetition frequency ranging from 0.3 to 3 Hz, preferably from 0.5 to 1 Hz.

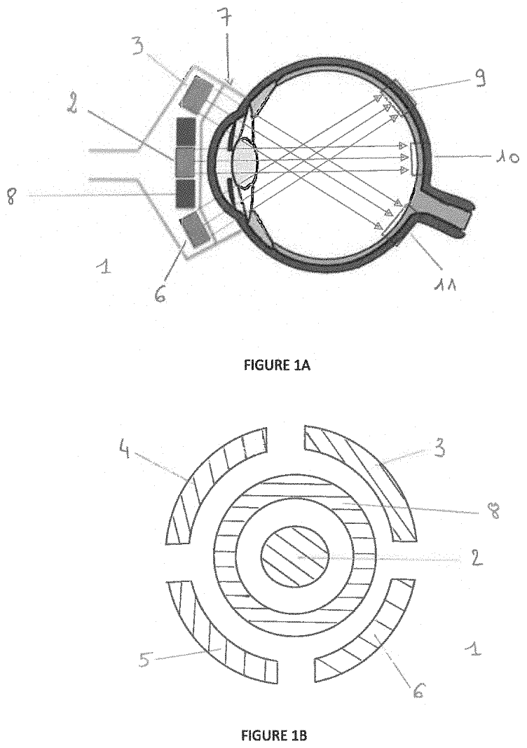

Advantageously, at least two unfocused US beams are applied sequentially to the retina. In such embodiment, each unfocused US beam targets a specific region of the retina, different from each other (see FIG. 1A). In a preferred embodiment, at least three, four, five, six, seven, eight, nine or ten unfocused US beams are applied sequentially to the retina, each one targeting a different region of the retina.

When several unfocused US beams are applied, the distance between the different targeted regions of the retina are preferably spaced apart from each other by at least 1 mm, preferably at least 2 mm. More generally speaking, the US beams are spaced apart from each other in order to avoid any beam superposition deposit onto the retina. Unfocused US beams target a specific region of the retina, different from each other. To avoid ultrasound superposition, specifically in the center of the aqueous humor, transducers are activated sequentially, so that they don't emit in the same time.

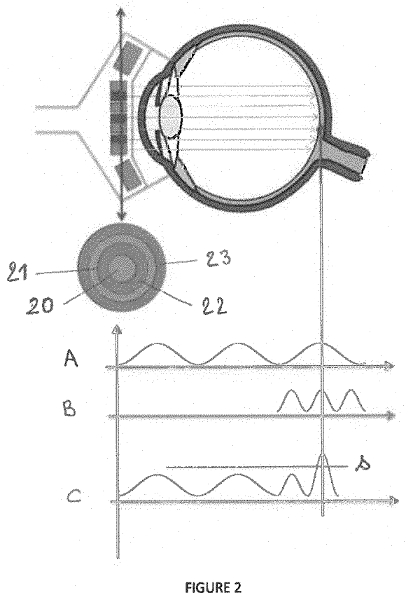

In another embodiment, the unfocused US beams are disposed concentrically and can be activated simultaneously in order to cumulate their acoustic effect on the retina and disrupting the BRB (see FIG. 2).

In order to preserve the anterior and middle chambers eye compartments from high/effective intensities of ultrasounds, and/or to avoid acoustic attenuation by the crystalline, the US transducers may emit at different frequencies (i.e., frequency modulation) and/or may start at different time (i.e., phase modulation) and/or emit with different pulse duration (i.e., pulse modulation).

The multiple unfocused US beams are applied sequentially on the retina. Advantageously the duration between two successive unfocused US beams is between 1 msec and 100 msec, preferably between 10 msec and 50 msec, more preferably, approximately 25 msec.

Advantageously, the unfocused US beams are applied for a short period of time, such as less than 5 minutes, preferably approximately 4 minutes.

### Ultrasound Contrast Agent

The method of the invention further requires the presence of an ultrasound contrast agent in the area of the BRB. The US contrast agent may be administered by injection, preferably by systemic injection. Systemic administration is a route of administration of an agent into the circulatory system so that the entire body is affected. Examples of systemic injections include intravenous, subcutaneous, intramuscular, intradermal, intravitreal, or perfusion.

In some embodiments, the ultrasound contrast agent is injected into the bloodstream of the subject.

Preferably, the ultrasound contrast agent is administered as a bolus just before the US beam application. More preferably, the US contrast agent is administered between 0 and 30 seconds before the US beam application. Advantageously, the US beam application and the US contrast agent administration are concomitant. When successive US beams are applied, the ultrasound contrast agent is preferably delivered only once, concomitantly with the first US beam application, though it may be delivered by a continuous infusion through the activation of successive US beams.

According to the invention, the ultrasound contrast agent may contain gaseous bubbles, a high concentration of gas, solid particles configured to vaporize in response to ultrasound, liquid configured to vaporize in response to ultrasound, micro particles configured to act as cavitation sites, solid particles having higher acoustic impedance than tissue in the desired region, and/or liquid with a high acoustic absorption coefficient.

In some embodiments, the ultrasound contrast agent is a microbubble contrast agent, preferably selected from the group consisting of sulphur hexafluoride microbubbles (SonoVue®), microbubbles made of an albumin shell and octafluoropropane gas core (Optison®), perflexane microbubbles encapsulated in an outer lipid shell amagent), microbubbles made of octafluoropropane gas core encapsulated in an outer lipid shell (Definity®), or perfluorobutaine and nitrogen gas encapsulated in a lipid shell (BR38—Schneider et al., 2011). Preferably, the ultrasound contrast agent consists of sulphur hexafluoride microbubbles.

The microbubbles may have a mean diameter in a range from 1 μm. to 20 μm. In some embodiments, the microbubbles have a mean diameter in a range from 4 μm to 5 μm. In some other embodiments, the microbubbles have a mean diameter in a range from 2 to 6 μm. In some embodiments, the microbubbles have a mean diameter of approximately 7 μm, 6 μm, 5 μm, 4 μm, 3 μm or 2 μm. In a particular embodiment, the microbubbles have a mean diameter of approximately 2.5 μm.

In some embodiments, the dose of ultrasound contrast agent ranges between 0.2 and 0.4 ml/kg based on the total weight of the subject. In a particular embodiment, the maximum dose of ultrasound contrast agent is up to 30 ml.

In some embodiments, a therapeutically active agent is used together with the ultrasound contrast agent. The therapeutically active agent is a drug that must be delivered to the retina of the patient. The therapeutically active agent is administered by injection, preferably by systemic injection.

In a particular embodiment, the therapeutically active agent and the ultrasound contrast agent are administered sequentially. The ultrasound contrast agent may be administered within a suitable time window prior to the administration of the therapeutically active agent. For example, the ultrasound contrast agent is administered less than 2 hours prior to the administration of the therapeutically active agent. Preferably, the ultrasound contrast agent is administered 5-120 minutes (e.g., 10-120, 10-110, 10-90, 10-60, 30-120, 30-90, or 30-60 minutes) prior to the administration of the therapeutically active agent.

In some embodiments, the ultrasound contrast agent is administered 10, 15, 20, 25, 30, 35, 40, 45 or 50 min prior to the administration of the therapeutically active agent. In one example, the ultrasound contrast agent is administered 10 minutes prior to the administration of the therapeutically active agent.

Alternatively, the ultrasound contrast agent and the therapeutically active agent may be administered concomitantly, or simultaneously, (e.g., by way of a same solution).

The “therapeutically active agent”, as used herein include any drug medicament, antibodies, glycoproteins, dissolution compounds, genetic materials such as RNA and DNA, stem cells, proteins or peptides, liposomes, lipids, synthetic or natural polymers or polymeric conjugates, macromolecules, nanocarriers, encapsulated drug molecules, pharmaceutical formulations, any other substance capable of producing therapeutic actions, and any mixtures thereof. In a particular embodiment, the therapeutically active agent is selected from growth factors, antibodies, stem cells, nanoparticles and liposomes.

Generally speaking, the use of an ultrasound contrast agent administered by injection to a subject, with the application of US beam to the retina of said subject, facilitates the delivery of any agent (endogenous or exogenous agent) across the BRB.

### BRB Disruption

The application of US beams to the retina of a subject in the presence of an ultrasound contrast agent injected to the subject leads to the transient opening of the BRB. In the context of the invention, a “transient” opening refers to a reversible opening/permeabilization occurring preferably for more than ½ hours, more preferably for more than 3 hours, the BRB returning after that to its initial state (i.e., the BRB state before the application of the first US beam).

In some embodiments, the BRB opening occurs for a period of time from 1 to 24 hours, preferably from 5 to 12 hours, more preferably from 6 to 10 hours. In some embodiments, the BRB opening occurs for approximately 8 hours.

The disruption may be confirmed and/or evaluated by fluorescent retinal angiography, magnetic resonance imaging (MRI), optical confocal microscopy, scanning laser ophthalmoscope. For example, a fluorescein-based retinal angiography dye such as Fluorescite® which does not normally cross the BRB, can be used to visualize the BRB disruption. When the fluorescent agent is intravenously injected, a photoluminescent digital camera can see fluorescein getting outside vessels of the retina. infrared Indocyanine angiography can visualize choroid vessels opening. With MRI, when Dotarem® (gadoterate meglumine, Guerbet USA) is injected in a patient, a T1w MR sequence can be used to visualize regions of hypersignal and therefore visualize the effect of BRB disruption by ultrasound. BRB disruption typically leads to a change of 5-10% or more in MR signal enhancement after contrast agent administration. In addition, dynamic contrast enhanced (DCE) MR imaging techniques can be used to calculate the permeability of the BRB and to quantify the magnitude of the permeability enhancement after ultrasound sonications.

Advantageously, an opening of the BRB refers to an opening of almost 65% of the BRB.

The BRB is then transiently disrupted, allowing to molecules, such as drugs, to cross it and to target the tissues of the retina. For instance, molecules already present in the subject's blood, and that may have potential therapeutic effects, can diffuse in the retina during and after the US application. Such natural molecules are for instance selected from albumin, endogenous antibodies, immune cells, etc. Alternatively or in addition, an exogenous therapeutically active agent may be administered to the subject in combination with the ultrasound contrast agent.

### Treatment of a Retina Disorder

The present invention relates to ultrasound contrast agent for use in treating a retina or choroid disorder by transiently disrupting the blood-retinal barrier (BRB) of a human, wherein the ultrasound contrast agent is administered before or during the application, to the retina/choroid of the human, of an unfocused ultrasound (US) beam.

The present invention also relates to therapeutically active agent for use in treating a retina and/or choroid disorder in a human, wherein the therapeutically active agent is to be delivered in combination with an ultrasound contrast agent, which is administered before or during the application, to the retina of the human, of an unfocused ultrasound (US) beam in order to transiently disrupting the blood-retinal barrier (BRB) of the human, to allow the therapeutically active agent to cross the BRB and to target the retina.

More generally speaking, the present disclosure provides a method for facilitating delivery of an agent (e.g., an endogenous or exogenous agent) across the BRB of a subject, comprising administering to a subject in need thereof ultrasound contrast agent prior to application of US beam to the retina of the subject.

The combined use of ultrasound contrast agent and US beam not only facilitates delivery of endogenous molecules (e.g., molecules that are naturally present in the blood stream of the subject) across the BRB, but also allows delivery of exogenous molecules (e.g., therapeutically active agents that are administered to the patient with the aim to target the retina), across the BRB. Systemic administration of ultrasound contrast agent within a suitable time window prior to the application of US beam to the retina of the subject temporarily increases the permeability of the BRB to these agents, thereby enhancing the delivery of the agents the retina.

Accordingly, provided herein are methods for enhancing treatment of a retina disorder by the co-use of ultrasound contrast agent and US beam, and optionally therapeutically active agent, wherein the ultrasound contrast agent may be systemically delivered within a suitable time window prior to the application of the US beam to the retina.

A method of treating a subject suffering from a retina disorder is also provided, which comprises: administering to the subject an ultrasound contrast agent within a suitable time window prior to the application of the US beam to the retina. Such method may be combined with the administration, in sequence or concomitantly with the ultrasound contrast agent, of a therapeutically active agent suitable to treat or prevent a retinal disorder.

The invention may be used for treating any kind of retina disorder that may be treated by delivery of a drug present in the blood stream. Preferably, the retinal disorder is selected from degenerative retinopathies, age related macular degeneration, diabetic retinopathy, hereditary retinal disorders and inflammatory retinal diseases.

### Eye Ultrasound Delivery Device

It is also the purpose of the invention to provide an eye ultrasound delivery device that may be used with ultrasound contrast agent for transiently disrupting the BRB.

The term “eye ultrasound delivery device”, as used herein, includes any apparatus capable of generating ultrasonic signals. For instance, the eye ultrasound delivery device comprises an electrical signal generator coupled with a power amplifier and US transducers. Electrical signals are emitted from the signal generator, amplified by the amplifier, and converted into mechanical ultrasonic signals in the US transducers, whereby US beams are produced.

According to the invention, the eye ultrasound delivery device comprises at least two unfocused plan US transducers disposed on a substrate suitable to be applied on the cornea of a human, wherein the unfocused plan US transducers can be activated sequentially. Both the transducer's shape and their sequential activation allow to avoid focalization, over application of US beams, and thereby allow to spare the eye compartment integrity.

In addition, in order to limit the risk of focalization of the US beams onto the retina, the US transducers are spaced apart from each other by at least 2 mm, preferably by at least 5 mm, on the substrate.

In a particular embodiment (FIGS. 1A and 1B), the eye ultrasound delivery device 1 comprises five unfocused plan US transducers 2, 3, 4, 5, 6. More particularly, the eye ultrasound delivery device 1 comprises a central US transducer 2 and four peripheral US transducers 3, 4, 5 and 6. The peripheral US transducers 3, 4, 5 and 6 are disposed circularly around the central US transducer 2. According to the invention, the US transducers can be activated sequentially to target different regions 9, 10, 11 the retina. The sequential activation of the transducers avoid focalization, over application of US beams, and allows to spare the anterior and middle eye compartments integrity. In order to further limit the risk of double exposition of the US beams onto the retina, the US transducers are advantageously spaced apart from each other by at least 1 mm, preferably by at least 2 mm, on the substrate 7. The position of the transducers on the substrate 7 is advantageously homogenous, to homogenously distribute the US beam on the surface of the retina.

According to the embodiment as illustrated in FIGS. 1A and 1B, the eye ultrasound delivery device 1 may further comprise a monitoring transducer 8, or US receptor (such as PVDF material), to monitor ultrasound contrast agent inertial cavitation and/or BRB opening. In this embodiment, the monitoring transducer 8 is circular and disposed concentrically between the central US transducer 2 and the peripheral US transducers 3, 4, 5, 6. The monitoring transducer 8 is advantageously placed in regards to the iris, i.e., between the central US transducer 2 and the peripheral US transducers 3, 4, 5, 6.

In an embodiment, the substrate is made of a flexible material able to perfectly match the contour of the eye of the subject. For instance, the substrate is a silicone substrate.

In another embodiment, a surface of the substrate, which must be applied on the eye of the subject, is coated with a degasified gel, silicon or fluid, in order to insure shape matching between the device and the eye, and ultrasound coupling. Therefore this allows for non-distortion of the US emission onto the eye, and to protect the cornea of any damages.

Alternatively or in addition, the eye US delivery device of the invention comprises immobilization means to insure adherence between the substrate and the eye. In a particular embodiment, the immobilization means comprise vacuum suction means. For instance, the eye US delivery device comprises a central vacuum suction chamber that allows the substrate by way of a suction cannula to perfectly adhere to the surface of the retina.

In a particular embodiment, the substrate further comprises at least one optical fiber, placed between two unfocused US transducers and/or in at least one unfocused US transducer, preferably in the middle of at least one unfocused US transducer.

Advantageously, the eye US delivery device is adapted to generate unfocused US beams with a resonance frequency ranging from 0.5 to 3 MHz, preferably at 1 MHz.

Advantageously, the eye US delivery device is adapted to generate unfocused US beams with a pressure level ranging from 0.3 to 2 MPa.

In another embodiment, the eye US delivery device comprises solely two transducers, one therapeutic active unfocused plan transducer to emit ultrasound to the retina, and a second large band monitoring passive transducer (US receptor) to monitor reflected ultrasound waves representative of the bubbles' cavitation (stable and inertial), respectively.

What is claimed is:

1. A method for treating a retina disorder by transiently disrupting the blood-retinal barrier (BRB) of a human,
wherein at least two unfocused ultrasound (US) beams are applied sequentially to the retina, the at least two unfocused US beams targeting different regions of the retina; and
wherein an ultrasound contrast agent is administered intravenously before, or during, or both before and during the application of unfocused US beam to the retina of the human; and
wherein the unfocused US beams are applied with an eye US delivery device comprising at least two unfocused plan US transducers disposed on a substrate suitable to be applied on the cornea of a human to target the retina of the human, wherein the unfocused US transducers are activated sequentially

2. The method for treating a retina disorder according to claim 1, wherein at least three, four, five, six, seven, eight, nine or ten unfocused US beams are applied sequentially to the retina, each one targeting a different region of the retina.

3. The method for treating a retina disorder according to claim 1, wherein the targeted regions of the retina are spaced apart from each other by at least 1 mm.

4. The method for treating a retina disorder according to claim 1, wherein the unfocused US beam is applied to the retina with a resonance frequency ranging from 0.5 to 3 MHz, wherein the unfocused US beam is applied in pulses of duration ranging from 10 to 300 ms and with a pulse repetition frequency ranging from 0.3 to 3 Hz, and wherein the unfocused US beam is applied with a pressure level ranging from 0.3 to 2 MPa.

5. The method for treating a retina disorder according to claim 1, wherein the dose of ultrasound contrast agent administered to the human is approximately 0.2 to 0.4 ml/kg with a maximum dose of up to 30 ml.

6. The method for treating a retina disorder according to claim 1, wherein a therapeutically active agent that must be delivered to the retina of the human is administered intravenously from 10 minutes before administration of the ultrasound contrast agent to 120 minutes after administration of the ultrasound contrast agent.

7. The method for treating a retina disorder according to claim 6, wherein the therapeutically active agent is selected from growth factors, antibodies, stem cells, nanoparticles and liposomes.

8. The method for treating a retina disorder according to claim 1, wherein the disorder is selected from degenerative retinopathies, age related macular degeneration, diabetic retinopathy, hereditary retinal disorders and inflammatory retinal diseases.

9. The method for treating a retina disorder according to claim 1, wherein the substrate comprises an annular unfocused plan US transducer, to be placed around the iris.

10. The method for treating a retina disorder according to claim 1, wherein the substrate further comprises at least one peripheral unfocused plan US transducer.

11. The method for treating a retina disorder according to claim 1, further comprising immobilization means to maintain the device on the retina of the human.

12. The method for treating a retina disorder according to claim 1, wherein the substrate further comprises at least one optical fiber, placed between two unfocused plan US transducers.

13. The method for treating a retina disorder according to claim 1, wherein the substrate further comprises at least one optical fiber, placed in at least one unfocused plan US transducer.

14. The method for treating a retina disorder according to claim 1, wherein the eye US delivery device generates unfocused US beams with a resonance frequency ranging from 0.5 to 3 MHz, and with a pressure level ranging from 0.3 to 2 MPa.

15. The method for treating a retina disorder according to claim 1, wherein the substrate further comprises at least 4 peripheral unfocused plan US transducers, disposed circularly around the central annular unfocused plan US transducer.
